(19) United States
(12) Patent Application Publication
(10) Pub. No.: US 2020/0319157 A1
(43) Pub. Date: Oct. 8, 2020

(54) METHOD AND A SYSTEM FOR FORECASTING TREE DISEASE USING MOISTURE INFORMATION

(71) Applicant: ECONNBIZ CO., LTD., Sejong-si (KR)

(72) Inventors: Yong Chan PARK, Goyang-si (KR); Seong Bean PARK, Suwon-si (KR); Chel Eung KIM, Hanam-si (KR)

(73) Assignee: ECONNBIZ CO., LTD., Sejong-si (KR)

(21) Appl. No.: 16/908,012

(22) Filed: Jun. 22, 2020

Related U.S. Application Data
(63) Continuation of application No. PCT/KR2018/007025, filed on Jun. 21, 2018.

(30) Foreign Application Priority Data
Dec. 20, 2017 (KR) 10-2017-0176107

(51) Int. Cl. G01N 33/00; G06Q 10/04; A01G 7/00

(52) U.S. Cl. CPC G01N 33/0098; G06Q 50/26; A01G 7/00; G06Q 10/04

(57) ABSTRACT

Disclosed are a method and a system for forecasting a tree disease using moisture information. The method is carried out by a server. The method includes receiving, by the server, moisture information measured from a tree, wherein the moisture information includes a plurality of moisture information measured from the tree over time, and performing, by the server, time-series analysis of the plurality of moisture information, wherein the server performs time-series analysis of moisture information of the tree as measured over a predetermined period of time, and determines that the larger a number of inflection points of a curve corresponding to the moisture information, the higher a probability at which the tree has been infected with the tree disease.

### CROSS-REFERENCE TO RELATED APPLICATIONS

The present application is a continuation of International Patent Application No. PCT/KR2018/007025, filed on Jun. 21, 2018 which is based upon and claims the benefit of priority to Korean Patent Application No. 10-2017-0176107 filed on Dec. 20, 2017. The disclosures of the above-listed applications are hereby incorporated by reference herein in their entirety.

### BACKGROUND

Embodiments of the inventive concept described herein relate to a method and a system for forecasting a tree disease using moisture information, and more specifically, a method and a system for performing time-series analysis of moisture information measured from a tree and forecasting a tree disease based on the analysis result.

There are various types of tree diseases. A pine wilt disease as a typical tree disease among these diseases refers to a disease in which pines dry and die due to pine wood nematode parasitic on a body of Monochamus alternatus as an insect vector or Monochamus saltuarius as a latent insect vector. A precursor symptom of the pine wilt disease may not be easily observed with a naked eye. A direction of movement of Monochamus alternatus carrying the pine wood nematode and propagation characteristics thereof have not been revealed to date. Moreover, when trees belonging to a pine family, such as Korean red pine, sea pine, Pinus Koraiensis, and Japanese white pine are infected with the pine wilt disease, 100% thereof dry and die. Thus, the pine wilt disease has been increasing damage every year since it was first identified at Busan Geumjeong Mountain in 1988.

In this situation, preventive activities are being performed to prevent the tree disease, but effect thereof is weak. There is still no clear way to fundamentally stop development of the tree disease. Accordingly, periodic aviation/ground chemicals spraying is done to block a proliferation pathway of the insect vector or the latent insect vector. Further, the insect vector capture is done. Moreover, control methods such as felling and fumigation of infected trees are being used to simultaneously remove the pine wood nematode, the insect vector and, the latent insect vector.

Currently, it is best to identify whether the tree disease has occurred and then take quick action accordingly. To this end, the present inventors devised the inventive concept forecasting the tree disease at high accuracy using various sensors, and analyzing data.

### SUMMARY

Prior art patent documents generally disclose a method for forecasting the pine wilt disease as the representative tree disease. In those patent documents, a forecasting method simply detects whether a sap flow inside a tree is smooth or not, and determines whether the pine wilt disease has occurred, based on the detection result. However, the drying and death of the tree due to the pine wilt disease is not simply due to clogging of a water stream of the tree. Thus, a new approach is needed to improve a forecasting accuracy of the pine wilt disease.

Embodiments of the inventive concept provide a method and a system for forecasting a tree disease in which time-series analysis of moisture information measured from a tree is performed to forecast the tree disease occurrence or non-occurrence based on the analysis result.

Embodiments of the inventive concept provide a method and a system for forecasting a tree disease in which moisture information over a predetermined period of time is analyzed and it is determined based on the analysis result that as a variation of the moisture information over time of a tree of interest is greater, the tree has a high probability at which the tree has been infected with the tree disease.

Embodiments of the inventive concept provide a method and a system for forecasting a tree disease in which moisture information is measured in a plurality of regions located in a single tree and the measured moisture information are compared with each other, and the tree disease occurrence or non-occurrence is determined based on the comparison result.

The technical purposes of the inventive concept are not limited to the technical purposes mentioned above. Other technical purposes as not mentioned will be clearly understood by those skilled in the art from following descriptions.

According to an exemplary embodiment, a method for forecasting a tree disease by a server includes receiving, by the server, moisture information measured from a tree, wherein the moisture information includes a plurality of moisture information measured from the tree over time, and performing, by the server, time-series analysis of the plurality of moisture information, wherein the server performs time-series analysis of moisture information of the tree as measured over a predetermined period of time, and determines that the larger a number of inflection points of a curve corresponding to the moisture information, the higher a probability at which the tree has been infected with the tree disease.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a schematic diagram of a tree disease forecasting system according to one embodiment of the inventive concept;

FIGS. 2A and 2B are schematic diagrams of a tree disease forecasting device according to various embodiments of the inventive concept;

FIG. 3 is a schematic diagram of a tree disease forecasting system according to another embodiment of the inventive concept;

FIG. 4 is a schematic diagram of a tree disease forecasting server according to one embodiment of the inventive concept;

FIG. 5 is a flowchart of a method for forecasting a tree disease according to one embodiment of the inventive concept;

FIGS. 6A and 6B are graphs showing moisture information of an abnormal tree over time, and FIG. 6C is a photograph of an abnormal tree located in a specific area;

FIGS. 7A and 7B are graphs showing moisture information of a normal tree over time, and FIG. 7C is a photograph of a normal tree located in a specific area; and

FIG. 8 is a graph showing a difference between moisture contents of the normal tree and the abnormal tree.

### DETAILED DESCRIPTION

Hereinafter, exemplary embodiments of the inventive concept will be described in detail with reference to the accompanying drawings. Advantages and features of the inventive concept, and methods of achieving them will become apparent with reference to embodiments described below in detail in conjunction with the accompanying drawings. However, the inventive concept is not limited to the embodiments disclosed below, but may be implemented in various forms. The present embodiments are provided to merely complete the disclosure of the inventive concept, and to merely fully inform those skilled in the art of the inventive concept of the scope of the inventive concept. The inventive concept is only defined by the scope of the claims. Like reference numerals refer to like elements throughout the disclosure.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The terminology used herein is for the purpose of describing the embodiments only and is not intended to limit the inventive concept. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or greater other features, integers, operations, elements, components, and/or portions thereof.

As used herein, a term “unit” may be a functional unit as implemented in software, hardware or a combination thereof.

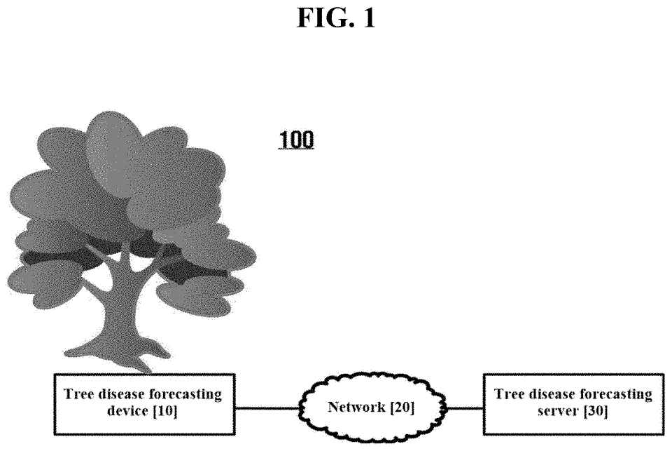

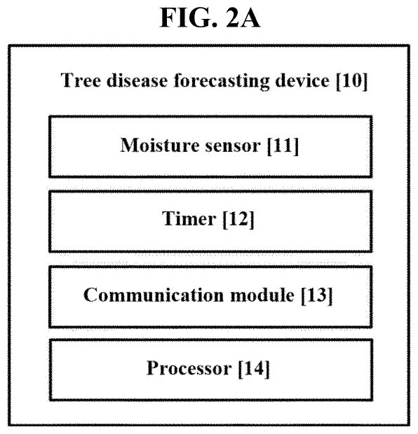

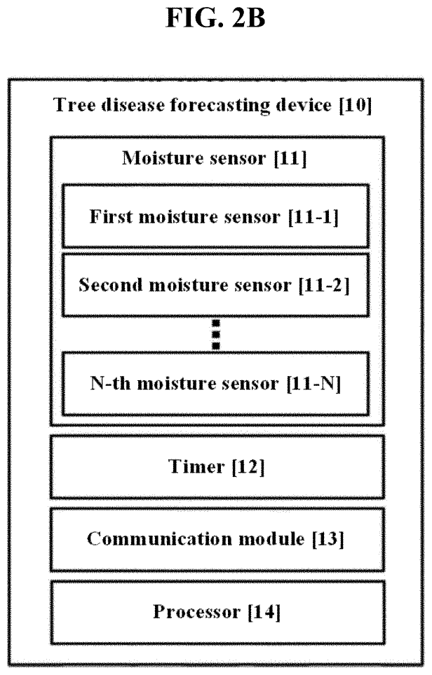

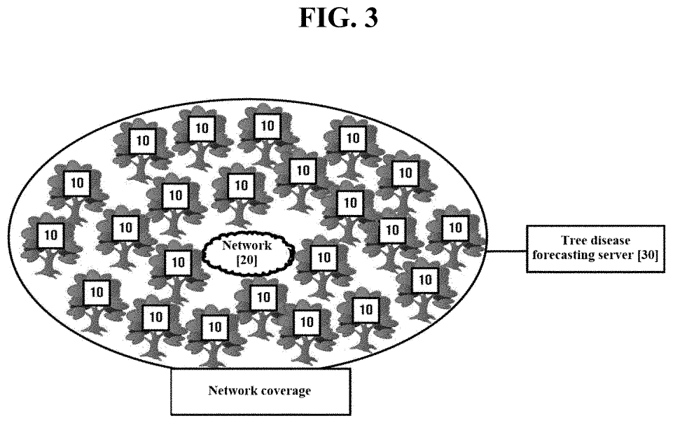

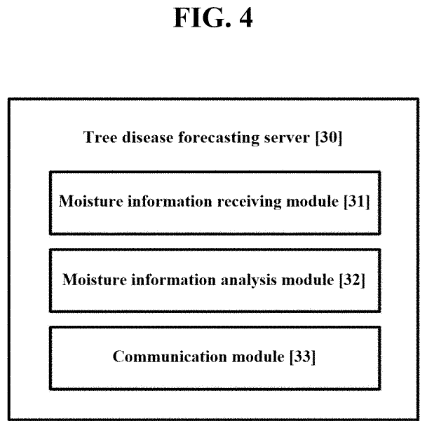

Hereinafter, with reference to FIG. 1 to FIG. 4, a tree disease forecasting system 100 according to one embodiment of the inventive concept is described. Referring to FIG. 1, a schematic diagram of the tree disease forecasting system 100 according to one embodiment of the inventive concept is shown. Referring to FIG. 2A and FIG. 2B, schematic diagrams of a tree disease forecasting device 10 according to various embodiments of the inventive concept are shown. Referring to FIG. 3, a schematic diagram of a tree disease forecasting system 100 according to another embodiment of the inventive concept is shown. Referring to FIG. 4, a schematic diagram of a tree disease forecasting server 30 according to one embodiment of the inventive concept is shown.

Referring to FIG. 1, the tree disease forecasting system 100 may include the tree disease forecasting device 10, a network 20 and the tree disease forecasting server 30. When the tree disease forecasting system 100 according to this embodiment is used, determination may be made as to whether a tree disease has occurred in a tree. The tree disease forecasting device 10 may collect information about the tree that may be a basis for determination of the tree disease. The tree disease forecasting server 30 may determine whether the tree of interest has been infected with the tree disease based on the information collected by the tree disease forecasting device 10. The tree disease forecasting device 10 and the tree disease forecasting server 30 may transmit and receive information to and from each other via the network 20.

However, in some embodiments, the tree disease forecasting device 10 not only may collect the information about the tree, but also may determine whether the tree of interest has been infected with the tree disease based on the collected information. In this case, the tree disease forecasting server 30 may be excluded from the tree disease forecasting system 100 or may receive the determination result from the tree disease forecasting device 10.

In this connection, a normal tree is a healthy tree. When the healthy tree has been infected with the tree disease, the healthy tree becomes an abnormal tree. The tree disease forecasting system 100 of this embodiment may forecast whether the tree has become an abnormal tree due to infection with the tree disease. The tree disease may be any of a variety of diseases that may occur in the tree. In the present disclosure, the pine wilt disease caused by the pine wood nematode parasitic on a body of Monochamus alternatus as an insect vector or Monochamus saltuarius as a latent insect vector is described as an example of the tree disease. Further, in the present disclosure, the tree may be, for example, a tree belonging to a pine family, such as Korean red pine, sea pine, Pinus Koraiensis, and Japanese white pine. However, the present disclosure is not limited thereto. The tree may belong to other tree families such as Japanese larch, Picea jezoensis, Abies holophylla, and Hymalaya Cedar.

Hereinafter, each of the components included in the tree disease forecasting system 100 according to this embodiment will be described in detail.

The tree disease forecasting device 10 may collect information related to the tree that may be the basis for determination of the tree disease. The collected information may be transmitted to the tree disease forecasting server 30 via the network 20. For example, the tree disease forecasting device 10 may include a moisture sensor 11, a timer 12, a communication module 13 and a processor 14. However, the present disclosure is not limited thereto. The tree disease forecasting device 10 may include more or fewer components than those illustrated in FIGS. 2A and 2B. That is, the tree disease forecasting device 10 may further include a sensor capable of measuring various factors such as a temperature and a humidity in addition to the moisture in order to collect various determination basis for the tree disease forecasting.

The moisture sensor 11 is placed in the tree to measure the moisture information about the tree. In this connection, the moisture information may include a moisture content. Specifically, the moisture sensor 11 may measure the moisture content in the tree using a heat pulse scheme. In this connection, the moisture sensor 11 may measure the moisture content in a trunk of the tree. To place the moisture sensor 11 inside the tree, a hole may be invasively drilled in the trunk of the tree and the moisture sensor 11 may be inserted into the corresponding hole.

In some embodiments, referring to FIG. 2B, the single tree disease forecasting device 10 may include a plurality of moisture sensors 11-1 to 11-n. The moisture sensors 11-1 to 11-n may be used respectively to measure moistures at various locations (regions) in one tree simultaneously. Alternatively, the moisture sensors 11-1 to 11-n may be used respectively to measure moistures of the tree and soil simultaneously, or to measure moistures of a plurality of trees simultaneously.

The timer 12 is configured for calculating time information, for example, may be embodied as a relay, but may not be limited thereto. The tree disease forecasting device 10 may use the time information of the timer 12 to specify a time at which the moisture information is measured by the moisture sensor 11, and then may send the time information along with the moisture information as collected to the tree disease forecasting server 30.

The communication module 13 may exchange information with an external device. For example, the moisture information measured from the moisture sensor 11 and the time information calculated by the timer 12 may be transmitted to the tree disease forecasting server 30 via the network 20.

The processor 14 may be configured to control an operation of the tree disease forecasting device 10, for example, to control a measurement period of the moisture sensor 11 or to control a transmission period at which the measured information is transmitted to the tree disease forecasting server 30 via the communication module 13. In some embodiments, the processor 14 may determine whether the tree has been infected with the tree disease based on the moisture information collected by the moisture sensor 11. However, the present disclosure is not limited thereto.

In one example, as shown in FIG. 3, each tree disease forecasting device 10 may be allocated to each of the trees. However, the present disclosure is not limited thereto. A plurality of moisture sensors 11-1 to 11-n may be included in one tree disease forecasting device 10, so that a plurality of trees may share one tree disease forecasting device 10.

As shown in FIG. 3, the tree disease forecasting system 100 may be applied not only to a small area where tens of trees are planted, but also to a mountain where thousands or tens of thousands of the trees are planted. The network 20 according to this embodiment may support various communication schemes. For example, the network 20 may use Wi-Fi, Bluetooth, LTE or LPWA (low power wide area) as well as existing communication networks. When the network 20 according to the present embodiment employs the LPWA communication scheme, the tree disease forecasting system 100 according to the present embodiment may be applied to a wide region such as a mountain.

With reference to FIG. 4, the tree disease forecasting server 30 included in the tree disease forecasting system 100 is described.

Referring to FIG. 4, the tree disease forecasting server 30 may determine whether a tree of interest is infected with a tree disease, based on the information collected by the tree disease forecasting device 10. The tree disease forecasting server 30 may include a moisture information receiving module 31, a moisture information analysis module 32 and a communication module 33. However, the tree disease forecasting server 30 may be composed of more or fewer components than those illustrated in FIG. 4.

The moisture information receiving module 31 may receive and store the information collected from the tree disease forecasting device 10. In addition, information necessary for forecasting occurrence or non-occurrence of the tree disease may be stored therein. For example, the moisture information receiving module 31 may receive and store the moisture information about the tree as measured from the moisture sensor 11 disposed in the tree, that is, the moisture information measured from the tree. In this connection, the moisture information may include a plurality of moisture information measured from the tree over time.

The moisture information analysis module 32 may forecast occurrence or non-occurrence of the tree disease based on the moisture information about the tree. The moisture information analysis module 32 may perform time-series analysis of the plurality of moisture information to determine a probability at which the tree of interest has been infected with the tree disease or to determine that the tree of interest is an abnormal tree infected with the tree disease.

Details of how the tree disease forecasting unit forecasts the tree disease will be described later.

The communication module 33 may exchange information with an external device. The information collected from the tree disease forecasting device 10 may be received by the communication module 33 via the network 20.

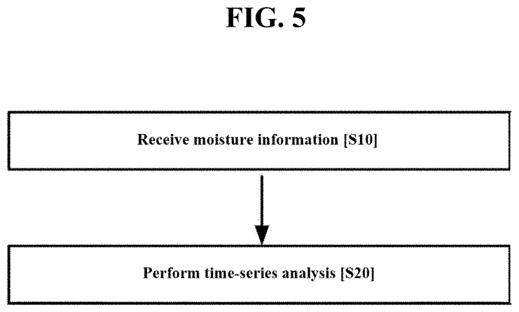

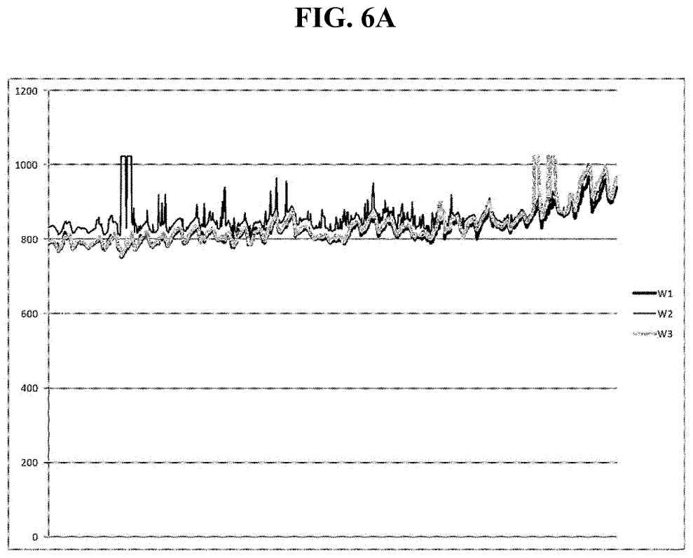

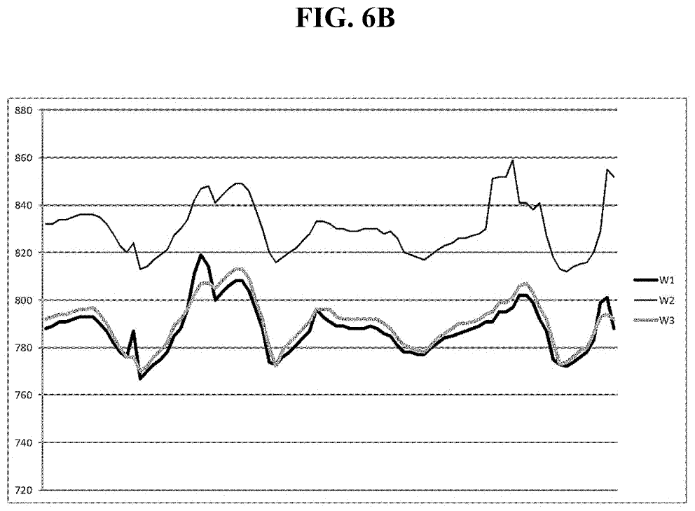

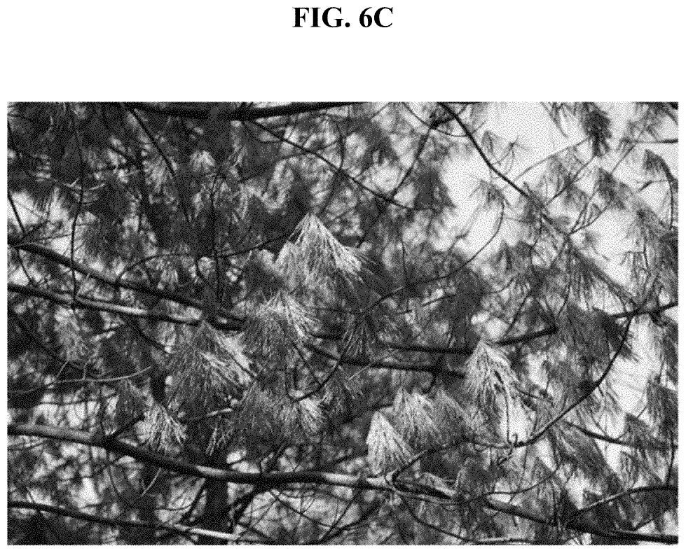

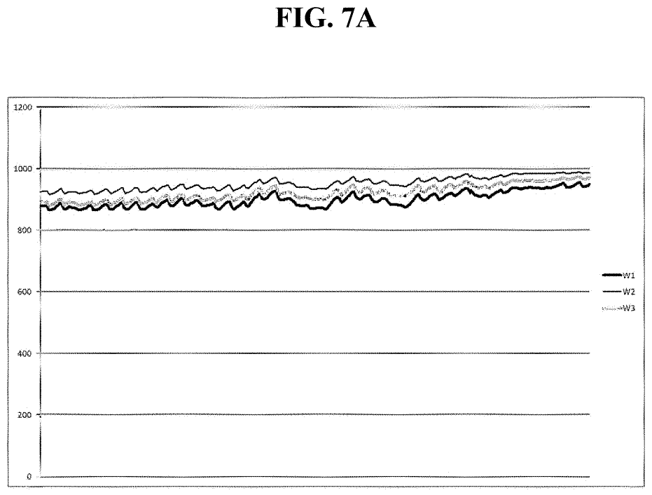

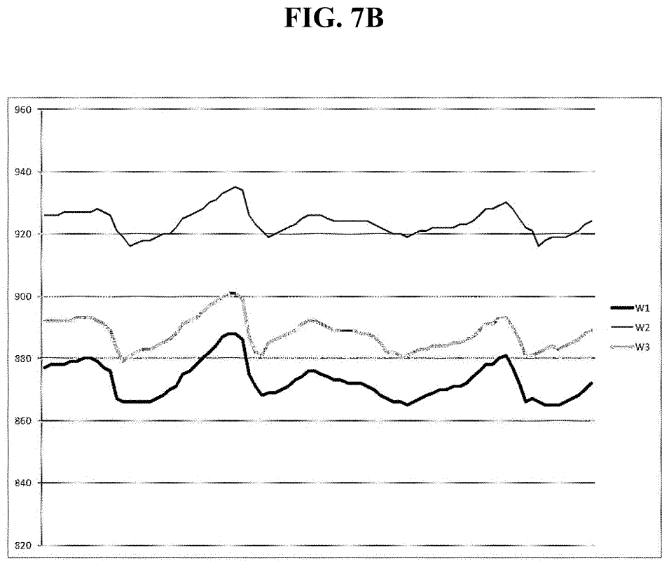

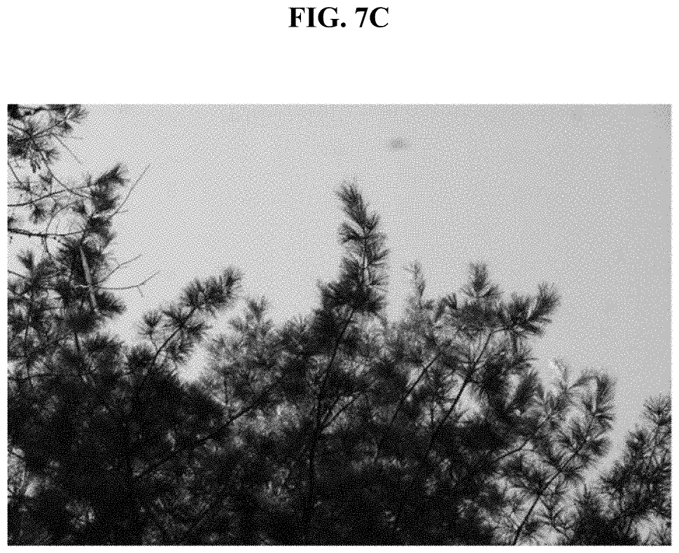

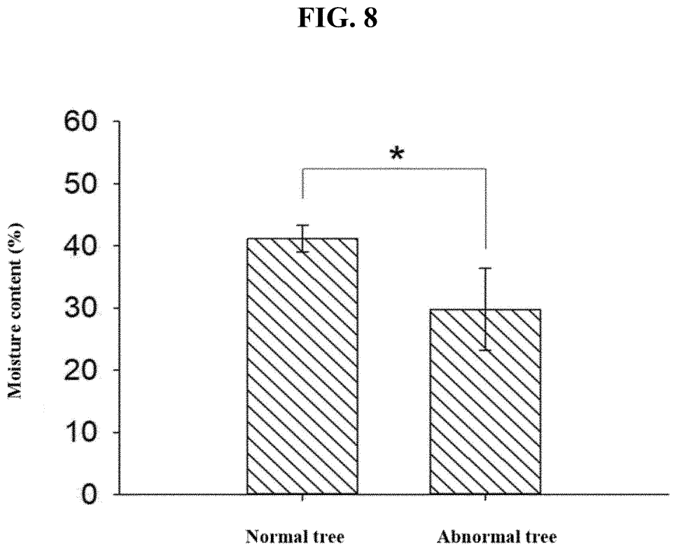

Hereinafter, a method for forecasting a tree disease using moisture information by the tree disease forecasting system 100 according to one embodiment of the inventive concept will be described with reference to FIG. 5 to FIG. 8. Referring to FIG. 5, a flowchart of a method for forecasting the tree disease according to one embodiment of the inventive concept is shown. Referring to FIGS. 6A and 6B, graphs showing moisture information about an abnormal tree over time is shown. Referring to FIGS. 7A and 7B, graphs showing the moisture information about a normal tree over time is shown. Referring to FIG. 8, a graph showing a difference between moisture contents of the normal tree and the abnormal tree is shown.

Specifically, FIG. 6A is moisture information for 1.5 months about an abnormal tree located in a specific area. FIG. 6B is moisture information for 4 days in FIG. 6A. FIG. 6C is a photograph of an abnormal tree located in a specific area. FIG. 7A shows moisture information for 1.5 months about a normal tree located in a specific area. FIG. 7B is moisture information for 4 days in FIG. 7A. FIG. 7C is a photograph of a normal tree located in a specific area. The abnormal tree in FIG. 6C and the normal tree in FIG. 7C were located in the same area, and the moisture information was measured during the same period.

Referring to FIG. 6C and FIG. 7C, it may be identified that there is a difference between leaf densities of the abnormal tree and the normal tree.

In FIG. 6A, FIG. 6B, FIG. 7A, FIG. 7B, an X-axis may represent a flow of time, and a Y-axis may indicate a resistance value as moisture information measured through the moisture sensor 11. In this connection, the measured resistance value may represent a value proportional to the moisture information.

In this connection, a subject for forecasting the tree disease may be the tree disease forecasting device 10 or the tree disease forecasting server 30. For example, when performing forecasting of the tree disease by the tree disease forecasting device 10, the processor 14 of the tree disease forecasting device 10 may perform a method of forecasting the tree disease using the moisture information. When performing forecasting of the tree disease by the tree disease forecasting server 30, the moisture information analysis module 32 of the tree disease forecasting server 30 may be used to forecast the tree disease using the moisture information. Herein, an example in which the tree disease forecasting server 30 forecasts the tree disease will be set forth. However, the method of forecasting the tree disease as described in the present disclosure may be applied equally to a case when the tree disease forecasting device 10 forecasts the tree disease.

First, referring to FIG. 5, the tree disease forecasting server 30 receives the moisture information measured from the tree (S10).

Specifically, the plurality of moisture information measured by the tree disease forecasting device 10 may be received via the moisture information receiving module 31 of the tree disease forecasting server 30 and may be stored in the tree disease forecasting server 30.

Because the tree disease forecasting device 10 includes the moisture sensor 11, the tree disease forecasting device 10 may be connected to the tree and may continuously measure the moisture information of the tree. The moisture information measured by the tree disease forecasting device 10 may include a plurality of moisture information measured from the tree over time. The information measured by the tree disease forecasting device 10 may be transmitted to the tree disease forecasting server 30 in real time. Alternatively, the information measured by the tree disease forecasting device 10 may be transmitted to the tree disease forecasting server 30 periodically or aperiodically via communication module 13.

However, in some embodiments, in order to increase a measurement time duration by minimizing a battery consumption of the tree disease forecasting device 10, the tree disease forecasting device 10 may be connected to the tree and may measure the moisture information of the tree not continuously but periodically.

Next, referring to FIG. 5, the tree disease forecasting server 30 performs time-series analysis of the plurality of moisture information (S20).

Specifically, the moisture information analysis module 32 may perform time-series analysis of the moisture information of the tree measured over a predetermined period and may determine a probability at which the tree of interest has been infected with the tree disease or determine that the tree of interest has been infected with the tree disease and becomes an abnormal tree. In other words, the present inventors invented a technique to perform the time-series analysis of the moisture information of the tree to forecast the tree disease. Thus, the abnormal tree may be identified at high accuracy. In this connection, the moisture information for at least 24 hours is required for accurate analysis. Thus, the predetermined period may be 24 hours or greater.

First, the moisture information analysis module 32 performs time-series analysis of the moisture information of the tree measured over the predetermined period of time. Then, the moisture information analysis module 32 determines that the larger a difference between the highest and lowest values of the moisture information, the greater the probability at which the tree has been infected with the tree disease. The method for forecasting the tree disease according to this embodiment may forecast the tree disease using the moisture information measured at a single location of the tree via the moisture sensor 11. In addition, the method for forecasting the tree disease according to this embodiment may forecast the tree disease without measuring moisture information at other locations in the tree using the moisture sensor 11.

In FIGS. 6A and 6B showing the moisture information about the abnormal tree over time and FIGS. 7A and 7B showing the moisture information about the normal tree over time, W1, W2 and W3 indicates moisture information. Based on W1, W2, and W3, the normal tree has a smaller variation in a level of the moisture information according to temporal change between day and night. However, the abnormal tree has a larger variation in a level of the moisture information according to temporal change between day and night.

In one example, a difference between heights of positions of the tree at which the moisture is measure by the moisture sensor 11 may be 100 cm or greater. For example, W1 is moisture information of the tree as measured by the moisture sensor 11 at a position spaced by a distance between 270 and 300 cm from a soil. W2 is moisture information of the tree measured by the moisture sensor 11 at a position spaced by a distance between 150 and 170 cm from a soil. W3 is moisture information of the tree measured by the moisture sensor 11 at a position spaced by a distance between 30 and 50 cm from a soil.

Therefore, the moisture information analysis module 32 performs time-series analysis of the moisture information of the tree, and identifies a graph pattern in which the larger the difference between the highest and lowest values of the moisture information, the larger the variation in the level of the moisture information, and determines that the tree of interest is more likely to be infected with the tree disease.

For example, when comparing W1 in FIG. 6B and W1 in FIG. 7B with each other, it may be identified that in FIG. 6B related to the abnormal tree, a difference between the highest and lowest values of the moisture information is about 50, while in FIG. 7B related to the normal tree, a difference between the highest and lowest values thereof is smaller than 20.

In some embodiments, the moisture information analysis module 32 performs time-series analysis of the moisture information of the tree as measured over a predetermined period of time. Then, when the difference between the highest and lowest values of the moisture information is greater than or equal to a predetermined value, the moisture information analysis module 32 determines that the tree of interest is an abnormal tree infected with the tree disease.

Second, the moisture information analysis module 32 performs time-series analysis of the moisture information of the tree as measured over a predetermined period of time and then determines that the higher the number of inflection points, the higher the probability at which the tree has been infected with the tree disease. According to the method for forecasting the tree disease according to the present embodiment, the tree disease may be forecasted using moisture information measured at a single location of the tree via the moisture sensor 11. In addition, forecasting of the tree disease may be realized even when moisture information is not measured at other locations in the tree via the moisture sensor 11.

That is, in a graph pattern in which the numerical variation of the moisture information according to the temporal change between day and night is large, the moisture information fluctuates over time. Thus, the fluctuation may be identified based on the number of inflection points of the curve of the moisture information of the graph. Therefore, the moisture information analysis module 32 performs time-series analysis of the moisture information of the tree and identifies that as the number of inflection points increases, the numerical variation of the moisture information is larger, thereby to determine that the tree of interest has a high probability at which the tree has been infected with the tree disease.

In some embodiments, the moisture information analysis module 32 performs time-series analysis of moisture information of the tree as measured over a predetermined period of time, and then determines that when the number of inflection points is smaller than or equal to a predetermined value, the tree of interest is not infected with the tree disease. For example, when performing time-series analysis of the moisture information of the tree measured for 24 hours, and there is one or no inflection point, it may be diagnosed that the tree of interest is a normal tree that is not infected with the tree disease.

Third, the moisture information analysis module 32 may perform time-series analysis of the moisture information of the tree as measured over a predetermined period of time and determine that the smaller an average level of the moisture information, the higher the probability at which the tree has been infected with the tree disease. According to the method for forecasting the tree disease according to the present embodiment, the tree disease may be forecasted using moisture information measured at a single location of the tree via the moisture sensor 11. In addition, forecasting of the tree disease may be realized even though moisture information is not measured at other locations in the tree via the moisture sensor 11.

Referring to FIG. 8, it was identified based on a result of analyzing moisture contents of wood chips collected from the normal tree and the abnormal tree, that the abnormal tree exhibited a lower moisture content than the normal tree did. Further, it was identified based on a result of conducting a student t-test to identify a statistical significance of a result, that a p-value was 0.0465<0.05, and thus a difference between the moisture contents of the normal tree and the abnormal tree was significant.

That is, because the abnormal tree has a lower moisture content than that of the normal tree, a level of the moisture information of the abnormal tree as measured by the moisture sensor 11 is also inevitably lower than that of the normal tree. Therefore, the moisture information analysis module 32 may perform time-series analysis of the moisture information of the tree as measured over a predetermined period of time and then may determine that the smaller an average level of the moisture information, the higher the probability at which the tree has been infected with the tree disease.

For example, it may be identified that in FIG. 6A, the average level of the abnormal tree is about 800, whereas in FIG. 7A, the average level of normal trees is about 900.

In some embodiments, the moisture information analysis module 32 performs time-series analysis of moisture information of the tree as measured over a predetermined period of time and then determines that when the average level of the moisture information is smaller than or equal to a predetermined level, the tree of interest is an abnormal tree infected with the tree disease. In this connection, the predetermined level as used may not be determined uniformly and may be determined with considering various factors such as a measurement date of the moisture information and an ambient temperature/humidity of the tree of interest.

However, the tree disease may be forecasted with considering only one of the three determination factors for forecasting the tree disease as mentioned above. Alternatively, the tree disease may be forecasted with considering two or more of the three determination factors simultaneously.

Therefore, the method of forecasting the tree disease according to one embodiment of the inventive concept may analyze the moisture information of the tree in time series and may distinguish between a normal tree and an abnormal tree based on the analysis result, thereby to forecast whether the tree of interest has been infected with the tree disease at high accuracy.

Hereinafter, a method for forecasting a tree disease using moisture information by the tree disease forecasting system 100 according to another embodiment of the inventive concept will be described with reference to FIG. 5 to FIG. 7C. However, a difference between the forecasting method of the tree disease according to the above embodiment of the inventive concept and the forecasting method of the tree disease according to this embodiment of the inventive concept is mainly described. The method of forecasting the tree disease according to this embodiment of the inventive concept is different from the forecasting method of the tree disease according to the above embodiment of the inventive concept in that moistures are measured from two regions of different heights in one tree in this embodiment.

First, referring to FIG. 5, the tree disease forecasting server 30 receives moisture information measured from the tree (S10).

Specifically, the moisture information receiving module 31 may receive first and second moisture information measured in a first region and a second region of the same tree at the same time, respectively. The first and second moisture information may include a plurality of first and second moisture information measured from the tree over time, respectively.

In this connection, a height from the soil to the first region and a height from the soil to the second region are different from each other. A difference between the heights of the first region and the second region may be 100 cm or greater. For example, the height from soil to first region may be 150 to 170 cm, and the height from soil to second region may be 30 to 50 cm. Alternatively, the height from the soil to the first region may be 270 to 300 cm and the height from the soil to the second region may be 30 to 50 cm. Alternatively, the height from soil to the first region may be 270 to 300 cm and the height from soil to the second region may be 150 to 170 cm. However, the present disclosure is not limited thereto.

Next, referring to FIG. 5, the tree disease forecasting server 30 performs time-series analysis of the plurality of moisture information (S20).

Specifically, the moisture information analysis module 32 performs time-series analysis of the plurality of first and second moisture information, and then determines that an occurrence frequency of an event when a level of the first moisture information is greater than a level of the second moisture information at a first measurement time-point, and a level of the second moisture information is greater than a level of the first moisture information at a second measurement time-point is higher, the likelihood at which the tree has been infected with the tree disease is high.

That is, the abnormal tree shows a graph pattern in which the numerical change of moisture information according to the temporal change between day and night is large. Referring to FIGS. 6A and 6B, in some cases, curves of the levels of the moisture information of W1, W2 and W3 cross each other. That is, referring to FIGS. 6A and 6B, for the first region and the second region having a height difference of 100 cm or greater therebetween, the curves of the levels of the first region's first moisture information and the second region's second moisture information cross with each other. Specifically, referring to FIG. 6B, the curves of the levels of the moisture information of W1 and W3 cross each other.

On the other hand, referring to FIGS. 7A and 7B, in the normal tree, an occurrence frequency of an event in which the curves of the levels of the moisture information of W1, W2 and W3 cross each other is zero. Thus, the tree disease forecasting server 30 performs time-series analysis of the plurality of first and second moisture information, and determines that when an occurrence frequency of an event in which the curves of the levels of the moisture information cross each other is high, the tree of interest has a high probability at which the tree has been infected with the tree disease.

In some embodiments, the moisture information analysis module 32 performs time-series analysis of the plurality of first and second moisture information, and then determines that when the level of the first moisture information is greater than the level of the second moisture information at the first measurement time-point at the first region and the second region having a height difference of 100 cm or greater therebetween, and the level of the second moisture information is greater than the level of the first moisture information at the second measurement time-point at the first region and the second region, the tree of interest is an abnormal tree infected with the tree disease.

Hereinafter, a method of forecasting a tree disease using moisture information by the tree disease forecasting system 100 according to still another embodiment of the inventive concept will be described with reference to FIG. 5 to FIG. 7C. However, a difference between the forecasting method of the tree disease according to the above embodiment of the inventive concept and the forecasting method of the tree disease according to this embodiment of the inventive concept is mainly described. The method of forecasting the tree disease according to this embodiment of the inventive concept is different from the forecasting method of the tree disease according to the above embodiment of the inventive concept in that moistures are measured from three different regions in one tree in this embodiment.

First, referring to FIG. 5, the tree disease forecasting server 30 receives moisture information measured from the tree (S10).

Specifically, the moisture information receiving module 31 may receive first to third moisture information respectively measured in first to third regions of the tree. The first to third moisture information may include a plurality of first to third moisture information measured from the tree over time, respectively. First to third heights from the soil to the first region, the second region and the third region respectively may be different from each other. The first height may be larger than the second height which may be larger than the third height. A difference between adjacent regions may be 100 cm or greater. For example, the first height from the soil to the first region may be 270 to 300 cm, the second height from the soil to the second region may be 150 to 170 cm, and the third height from the soil to the third region may be 30 to 50 cm. However, the present disclosure is not limited thereto.

Next, with reference to FIG. 5, the tree disease forecasting server 30 analyzes time-series of the plurality of moisture information (S20).

Specifically, the moisture information analysis module 32 performs time-series analysis of the plurality of first to third moisture information, and determines that as a difference between a first difference of levels of the first moisture information and the second moisture information and a second difference between levels of the second moisture information and the third moisture information is smaller, the tree has a higher probability at which the tree has been infected with the tree disease. In this connection, the first difference between the levels of the first moisture information and the second moisture information may be compared with the second difference between the levels of the second moisture information and the third moisture information. In another example, a ratio between the first and second differences may be calculated as a difference between the first and second differences.

That is, referring to FIGS. 6A and 6B, in the abnormal tree, values of W1 and W3 are close to each other, and thus a difference between W1 and W2 and a difference between W2 and W3 are substantially equal to each other. Therefore, the tree disease may be forecasted using a difference between moisture information measured in upper and lower positions in the same tree. Specifically, a numerical difference between W1 and W2 and a numerical difference between W2 and W3 may be analyzed and then whether the tree of interest is an abnormal tree, based on the analysis result. Therefore, in the method for forecasting the tree disease according to this embodiment, the time-series analysis of the plurality of first to third moisture information may be performed, and then it may be determined that as a difference between the first difference of levels of the first moisture information and the second moisture information and the second difference between levels of the second moisture information and the third moisture information is smaller, the tree has a higher probability at which the tree has been infected with the tree disease.

On the other hand, referring to FIGS. 7A and 7B, it may be identified that a difference between the numerical difference between W1 and W3 and a numerical difference between W2 and W3 is relatively larger compared to that of the abnormal tree of FIGS. 6A and 6B.

In some embodiments, referring to FIGS. 7A and 7B, in the normal tree, the curve of the moisture information exhibits a flat change trend, and a spacing between the moisture levels from the plurality of moisture sensors is relatively small. Referring to FIGS. 6A and 6B, in the abnormal tree, the curve of the moisture information exhibits a sharp change trend, and a spacing between the moisture levels from the plurality of moisture sensors is relatively large. Thus, the method may determine whether the tree is highly likely to be infected with the tree disease based on the above trend and spacing.

In the example, when the tree disease forecasting system 100 forecasts the tree disease, the tree of interests is determined to be an abnormal tree or to have a high probability at which the tree is an abnormal tree. In this case, the tree disease forecasting system 100 may provide an alarm to a user who manages the tree of interest, such that the tree disease may be prevented from further spreading.

According to the inventive concept as described above, the following effects may be obtained. The effect that may be obtained via the inventive concept is not limited thereto.

First, according to the inventive concept, because the moisture information of the tree may be analyzed in time series and then the normal tree and abnormal tree may be distinguished from each other based on the time series analysis result. Thus, whether the tree of interest has been infected with the tree disease may be forecast at high accuracy.

Second, according to the inventive concept, because only moisture information of the tree is used for the tree disease forecasting, a relatively simplified tree disease forecasting system may be constructed.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A method for forecasting a tree disease by a server, the method comprising:
receiving, by the server, moisture information measured from a tree, wherein the moisture information includes a plurality of moisture information measured from the tree over time; and
performing, by the server, time-series analysis of the plurality of moisture information, wherein the server performs time-series analysis of moisture information of the tree as measured over a predetermined period of time, and determines that the larger a number of inflection points of a curve corresponding to the moisture information, the higher a probability at which the tree has been infected with the tree disease.

2. The method of claim 1, wherein the predetermined period of the time is 24 hours or greater.

3. The method of claim 1, wherein the performing of the time-series analysis includes performing the time-series analysis of the moisture information of the tree over the predetermined period, and determining that a larger a difference between highest and lowest values of the moisture information, the higher the probability at which the tree has been infected with the tree disease.

4. The method of claim 1, wherein the performing of the time-series analysis includes performing time-series analysis of the moisture information of the tree over the predetermined period, and determining that the smaller an average level of the moisture information, the higher the probability at which the tree has been infected with the tree disease.

5. The method of claim 1, wherein the tree disease includes a pine wilt disease.

6. A method for forecasting a tree disease by a server, the method comprising:
receiving, by the server, first and second moisture information measured in a first region and a second region of a specific tree, respectively, wherein the first moisture information includes a plurality of first moisture information measured from the specific tree over time, and the second moisture information includes a plurality of second moisture information measured from the specific tree over time; and
performing, by the server, time-series analysis of the plurality of first moisture information and the plurality of second moisture information,
wherein the performing of the time-series analysis includes:
calculating an occurrence frequency of an event in which a level of the first moisture information is greater than a level of the second moisture information at a first measurement time-point, and a level of the second moisture information is greater than a level of the first moisture information at a second measurement time-point; and
determining, based on the occurrence frequency, whether the specific tree has a high probability at which the tree has been infected with the tree disease or whether the specific tree has been infected with the tree disease,
wherein a height from soil to the first region is different from a height from the soil to the second region.

7. The method of claim 6, wherein the performing of the time-series analysis includes:
when the occurrence frequency is high, determining that the specific tree has a high probability at which the tree has been infected with the tree disease.

8. The method of claim 6, wherein the performing of the time-series analysis includes:
when the event occurs, determining that the specific tree has been infected with the tree disease.

9. The method of claim 8, wherein the performing of the time-series analysis includes:
when a state in which a level of the first moisture information is greater than a level of the second moisture information at the first measurement time-point changes to a state in which a level of the first moisture information is smaller than a level of the second moisture information at the second measurement time-point,
determining that the specific tree has been infected with the tree disease.

10. The method of claim 6, wherein a difference between heights of the first region and the second region is in a range of 100 cm or greater.

11. The method of claim 6, wherein the tree disease includes a pine wilt disease.

12. A system for forecasting a tree disease, the system comprising:
a moisture information receiving module configured to receive first and second moisture information measured in a first region and a second region of a specific tree, respectively, wherein the first moisture information includes a plurality of first moisture information measured from the specific tree over time, and the second moisture information includes a plurality of second moisture information measured from the specific tree over time; and
a moisture information analysis module configured to perform time-series analysis of the plurality of first moisture information and the plurality of second moisture information,
wherein the moisture information analysis module is further configured to:
calculate an occurrence frequency of an event in which a level of the first moisture information is greater than a level of the second moisture information at a first measurement time-point, and a level of the second moisture information is greater than a level of the first moisture information at a second measurement time-point; and
determine, based on the occurrence frequency, whether the specific tree has a high probability at which the tree has been infected with the tree disease or whether the specific tree has been infected with the tree disease,
wherein a height from soil to the first region is different from a height from the soil to the second region.

13. The system of claim 12, wherein the moisture information analysis module is further configured to:
when the occurrence frequency is high, determine that the specific tree has a high probability at which the tree has been infected with the tree disease.

14. The system of claim 12, wherein the moisture information analysis module is further configured to:
when the event occurs, determine that the specific tree has been infected with the tree disease.

15. The system of claim 14, wherein the moisture information analysis module is further configured to:
when a state in which a level of the first moisture information is greater than a level of the second moisture information at the first measurement time-point changes to a state in which a level of the first moisture information is smaller than a level of the second moisture information at the second measurement time-point,
determine that the specific tree has been infected with the tree disease.

16. The system of claim 12, wherein a difference between heights of the first region and the second region is in a range of 100 cm or greater.

17. The system of claim 12, wherein the tree disease includes a pine wilt disease.
